(19) United States
(12) Patent Application Publication
(10) Pub. No.: US 2022/0125354 A1
(43) Pub. Date: Apr. 28, 2022

(54) PHYSICAL FITNESS TRAINING SYSTEMS AND METHODS

(71) Applicant: Abbott Diabetes Care Inc., Alameda, CA (US)

(72) Inventors: Randy Shao Chin Shen, Sunnyvale, CA (US); Judd R. Van Sickle, Sacramento, CA (US)

(73) Assignee: Abbott Diabetes Care Inc., Alameda, CA (US)

(21) Appl. No.: 17/309,197

(22) PCT Filed: Oct. 25, 2019
(86) PCT No.: PCT/US2019/058067

§ 371 Date: May 6, 2021

Related U.S. Application Data
(60) Provisional application No. 62/757,451, filed on Nov. 8, 2018.

(51) Int. Cl. A61B 5/145; A61B 5/1486; A63B 24/00; G16H 20/30; G16H 10/40; G16H 50/30

(52) U.S. Cl. CPC A61B 5/14546; A61B 5/14865; A63B 24/0062; A61B 5/4866; G16H 10/40; G16H 50/30; G16H 20/30

(57) ABSTRACT

In vivo lactate concentrations may be monitored to determine a rate of lactate clearance as one metric of an individual's physical fitness. The rate of lactate clearance may guide further training. Lactate may be monitored using a sensing system comprising a lactate-responsive sensor, and a signal from the sensor may be communicated to a processor for calculating the lactate concentrations and a rate of lactate clearance. Some methods for determining physical fitness may comprise: measuring a plurality of lactate concentrations in a biological fluid with a sensing system comprising a lactate-responsive sensor over a period of time while the lactate concentrations are decreasing following a peak lactate level; and specifying that a second exercise event be conducted after a recovery period in which the lactate level has fallen to a predetermined concentration.

### STATEMENT REGARDING FEDERALLY SPONSORED RESEARCH OR DEVELOPMENT

Not applicable.

### BACKGROUND

Data-driven training protocols are becoming increasingly important for both world-class athletes and others interested in improving their physical fitness and athletic performance. Aside from how well an activity is actually performed by an individual, numerous metrics may be analyzed to determine the individual's physical fitness such as, for example, heart rate, maximal oxygen uptake (VO₂ max), and lactate levels. Certain values may be measured during a workout routine or training protocol, such as heart rate and VO₂ max, which may be monitored using external sensors or equipment, some of which may be easier to operate than are others. External sensors are available for determining lactate levels as well, but the results are often rather inaccurate. Invasive blood draws may be required to obtain more reliable lactate levels, but this approach may be inconvenient or painful to perform for an individual during a workout routine or training protocol. Moreover, even rapidly performed blood draws (e.g., from the ear lobe) may require at least a brief stoppage in an individual's workout routine, which may lead to a sub-optimal workout efficiency.

Lactate is produced in vivo during exercise or other activities through glycolytic conversion of glucose, particularly during intense physical activity or exercise. Glycolysis supplies energy to help an individual maintain their current activity level. Lactate levels in an individual are typically characterized as residing within three different zones, as shown in FIG. 1. At lower activity levels (intensities), the rates of lactate production and lactate clearance remain fairly balanced with one another, such that lactate levels remain relatively constant at or near a fixed baseline concentration, possibly with a slight concentration rise (Zone I). In Zone I, lactate production and lactate clearance (use) change in proportion to one another at the muscular level. The baseline concentration may vary between different individuals. Once higher activity levels are reached, the rate of lactate production outstrips the rate of lactate clearance at the muscular level and blood lactate levels begin to rise (Zone II). In Zone II, the full body rate of lactate utilization may still be able to keep pace with the rate of production, such that blood lactate levels remain elevated but stable with sustained physical activity. At still higher activity levels, a pronounced and continued increase in blood lactate levels occurs upon continued physical activity at the same level or a higher level (Zone III). In Zone III, the full body rate of lactate utilization is no longer able to keep pace with the rate of production. The intensity of a physical activity at which lactate production exceeds lactate clearance and a pronounced and continued increase in lactate levels occurs is referred to as the “lactate threshold.” Like baseline lactate concentrations, the lactate threshold may vary between different individuals depending upon their specific fitness level and/or other individual factors. The lactate threshold of an individual may be altered through training or lack thereof. When increased athletic performance is desired, for example, training protocols may be instituted so that the lactate threshold occurs at a higher intensity than before the training took place. Training may likewise improve the rate of lactate clearance during a recovery period following exercise, and the rate of lactate clearance may also be diagnostic of an individual's physical fitness.

A change in an individual's lactate threshold and/or rate of lactate clearance may be diagnostic of their physical fitness. Outside of a sports training lab, it can presently be very difficult for an individual or their instructor to determine where the individual's lactate threshold lies. Even within the controlled environment of a sports training lab, there are often regimented training procedures that are followed to measure an individual's lactate threshold indirectly rather than relying upon the direct measurement of lactate concentrations from blood. Blood draws (e.g., from the ear lobe) may be conducted with minimal stoppage of an individual's workout routine, but the data collection frequency may be insufficient to determine the individual's lactate threshold accurately in many instances. Moreover, blood sample analyses are generally too slow to allow real-time modification of a training protocol so that the lactate threshold or the rate of lactate clearance may be impacted in a desired way.

Peak performance of an individual during an athletic event may occur when the individual's activity level is constrained to provide a specified, individually determined level of lactate, which may correspond to an intensity just below the lactate threshold in some instances. This approach may allow the individual to perform at their highest possible activity level and for the longest possible period of time without accumulating significant lactate in their muscles, thereby requiring a decrease in intensity or a rest period to allow excess lactate to clear. Like the lactate response to exercise, the rate of lactate clearance following a given physical activity may be diagnostic of an individual's physical fitness level.

For training purposes, particularly to increase an individual's lactate threshold and/or rate of lactate clearance, it may be desirable to perform an activity at high levels over short bursts, such that the lactate threshold or other predetermined lactate concentration is exceeded periodically, sometimes to a significant degree. Periodically exceeding the lactate threshold may increase an individual's tolerance toward the physical activity and increase the activity level at which onset of the lactate threshold occurs. The periodic performance of a given training activity at the same intensity or at a different intensity, particularly a training activity that sometimes results in the lactate threshold being exceeded, is referred to as interval training.

An important component of interval training is the degree of recovery experienced by an athlete, which can be measured by the rate at which an individual clears excess lactate after the cessation of each interval. In order for interval training to be most effective, an individual needs a sufficient recovery time to clear at least a portion of the excess lactate after performing a given training activity. Otherwise, the individual may be unable to perform a subsequent interval at a sufficiently high activity level or for a sufficient length of time to achieve a desired training goal. Conversely, waiting too long to perform a subsequent interval may result in sub-optimal training as well.

Due to differing fitness levels and innate physiological variability, accurate determination of the recovery period for a given individual can be difficult to ascertain. Various individuals, even world-class athletes, may recover at considerably different rates post-exertion. Depending on the type of activity, its intensity, how long the activity was performed, and the intensity of the recovery activity, the recovery period can vary from minutes to hours before a subsequent interval may be most effectively performed. Such variability can make it especially difficult to tailor a training protocol for a given individual. Moreover, since the recovery period itself may vary as an individual's physical fitness or level of fatigue changes, it can be difficult to maintain an extended interval training program at a high level of efficiency. Accordingly, an individual may need to alter the way in which they train on an ongoing basis in order to continue to improve performance. At present, there is a delay in optimizing interval training efficiency due to the speed at which physiological data is collected and analyzed.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 shows an illustrative plot of lactate levels as a function of variable intensity physical activity.

FIG. 2A shows a diagram of an illustrative sensing system that may incorporate a lactate-responsive sensor of the present disclosure. FIG. 2B shows a block diagram of a processing electronics that may be associated with one or more components of the sensing system.

FIG. 3A shows a diagram of an illustrative two-electrode sensor configuration compatible for use in the disclosure herein.

FIG. 3B shows a diagram of an illustrative three-electrode sensor configuration compatible for use in the disclosure herein. FIG. 3C shows a diagram of another configuration of an illustrative three-electrode sensor configuration compatible for use in the disclosure herein.

FIG. 4 shows an illustrative plot of lactate concentration as a function of time for a single exercise event.

FIG. 5 shows an illustrative plot demonstrating how baseline concentrations of lactate, peak lactate levels, time to reach peak lactate levels, and rates of lactate clearance may vary between different individuals in a single exercise event.

FIG. 6 shows an illustrative plot showing how parameters of a second exercise event may be adjusted in response to the rate of lactate clearance following a first exercise event.

FIGS. 7A-7C show illustrative plots demonstrating how lactate levels may respond to altering the intensity, duration, or timing of subsequent exercise events in a plurality of exercise events.

FIG. 8 shows the output of a lactate-responsive sensor as compared to lactate concentrations measured via an external infrared spectroscopy technique.

FIG. 9 shows the output of a lactate-responsive sensor as compared to heart rate in a typical progressive exercise test.

FIG. 10 shows the output of two different lactate-responsive sensors worn in different locations during a cycling event.

FIG. 11 shows the lactate sensor response as a function of time for two different lactate-responsive sensors implanted at different depths.

### DETAILED DESCRIPTION

The present disclosure generally describes methods for assessing recovery from a physical activity and, more specifically, methods and systems for monitoring lactate levels in vivo for optimizing physical fitness training.

Lactate concentrations of an individual may change dynamically in response to various physiological and environmental factors. Glycolytic production of lactate during a physical activity is one route through which lactate levels may undergo significant dynamic variation. Tolerance toward increased lactate levels, the onset activity level at which increased lactate levels occur, and the rate of lactate clearance may be diagnostic of an individual's physical fitness or changes thereof. Despite the wealth of physical fitness information that may be gleaned from monitoring lactate levels in an individual, it is presently difficult to do so with sufficient regularity and accuracy to make meaningful decisions for modifying a training routine. It can be particularly cumbersome to measure lactate levels on an ongoing basis while lactate concentrations are decreasing following a peak lactate level (i.e., during recovery following an interval), even with the resources of a sports training facility, especially given the significant variability in the rate of lactate clearance between different individuals and their tolerance to particular lactate levels. This difficulty may increase further once the athlete undertakes activities beyond the confines and resources of a sports training facility.

Both excessively long and excessively short lactate clearance periods may be problematic. For short lactate clearance periods, traditionally determined (i.e., laboratory) measurements of lactate levels may be insufficient for accurately assessing and proactively altering a physical fitness routine. In the case of long lactate clearance periods, it may be impractical for an individual to remain in a sport training facility or a medical facility for ongoing blood draws or other types of lactate monitoring in order to follow lactate clearance to completion or some other predetermined value. Intermediate length lactate clearance periods may be less problematic, but it may still be desirable to collect further lactate concentration data than may be practically obtained through existing techniques. Even at intermediate length lactate clearance periods, cessation of physical activity and lack of user autonomy may be impediments for collecting lactate clearance data. Moreover, it may be logistically impractical for an athlete to perform all of their training in the confines of a laboratory environment.

In contrast, analyte sensors that are responsive to lactate in vivo (also referred to herein as “lactate sensors” or “lactate-responsive sensors,” any of which are to be understood to be capable of providing “continuous” measurement of lactate concentrations) may be operable to provide a plurality of lactate concentrations over extended (continuous) periods of time, such as hours to days or weeks. Lactate sensors may provide a number of advantages for monitoring dynamic lactate levels associated with a physical activity, particularly when incorporating in a sensing system as described further herein. Lactate sensors may allow lactate levels to be measured with sufficient frequency and accuracy to allow proactive training decisions to be made on-the-fly during a fitness routine. An individual wearing the sensor may make a decision based upon lactate levels how to conduct a subsequent exercise event, or such a decision may be made under the direction of a trainer or other individual not wearing the sensor. Sensor electronics and processing algorithms associated with operating the sensors and sensing systems, including display units or devices thereof, may also provide direction, guidance, recommendations and/or output concerning lactate concentrations and how a subsequent exercise event should be performed in order to meet a specified training goal, as described further hereinafter. Suitable processing algorithms, processors, memory, electronic components, and the like may reside in any of a trusted computer system, remote terminal, cloud server, reader device, and/or a housing for the sensor itself. Guidance, recommendations, output and/or the like may be shown upon a suitable display unit or device that is in electronic communication with one or more of these components. The display unit or device may be a dedicated reader device. Alternately, the display unit or device may be a third-party server, cloud server, or remote terminal that communicates with various software- and healthcare-related applications, such as a FITBIT or other personal health monitor. Suitable remote terminals, cloud servers, or the like may further relay an output to associated secondary devices such as smart home devices, wearable technology, personal health monitors, or the like.

Moreover, a single lactate sensor may be used to measure the lactate concentration multiple times over the course of a given fitness routine, thereby removing a potential source of variability associated with conventional laboratory measurements. In vivo lactate monitoring using a lactate sensor may be particularly advantageous for observing lactate levels during the recovery period following an exercise event, such as an exercise event in which a lactate threshold has been approached or exceeded and excess lactate is being cleared. The significant variability of lactate clearance rates among individuals, including their responses to different types of physical activity, may make ongoing laboratory-based monitoring of lactate levels difficult. Lactate sensors, in contrast, may overcome the challenges associated with both long and short lactate clearance periods. As such, employing a lactate sensor to monitor an individual's lactate clearance may allow their physical fitness to be both better observed and managed more effectively. More specifically, by monitoring the rate of lactate clearance in an individual, subsequent exercise events may be altered to affect a training outcome in a desired fashion.

Lactate clearance rates may be representative of an individual's fitness level. Faster clearance following an intense bout of exercise may indicate that whole body lactate utilization is improved, which may be one indicator of an individual's physical fitness. Since blood lactate levels represent a balance of production and clearance, an improved lactate clearance rate may either reduce lactate for a given exertion level, or enable a greater sustainable glycolytic contribution to exercise before the onset of fatigue, which may be characterized by unsustainable lactate levels. If several exercise events (intervals) are being conducted, one may measure the time it takes from the start of one or more of the intervals to achieve intermediate peak lactate values, thus indicating the changes occurring in the rates of overall lactate production and metabolic utilization. As fatigue accumulates, the time required to reach peak lactate values may shorten with each successive interval.

The present disclosure further describes sensing systems incorporating a lactate sensor that may facilitate the above. The systems may include various sensing components, such as a processor and/or coding instructions (algorithms) therein, that are adapted to process sensor data received from the lactate sensor and determine a plurality of lactate concentrations therefrom. The processor and/or coding instructions may then analyze the lactate concentrations to determine a rate of lactate clearance and, in some embodiments, further analyze the rate of lactate clearance or other quantity derivable from the lactate concentrations to suggest an appropriate training protocol for achieving a desired fitness outcome. In the case of interval training, for example, the processor and/or coding instructions may suggest various ways in which a subsequent exercise event should be conducted to achieve a desired fitness outcome, such as changing the timing (i.e., when), duration (i.e., length), and/or intensity (i.e., power) of an exercise event to be performed at some point in the future. For example, depending upon an individual's specific training goals, a subsequent exercise event may be conducted once lactate levels have fallen below a peak lactate level, to a baseline concentration, or somewhere in between. Peak lactate levels can be the overall highest recorded lactate concentration (e.g., a blood lactate concentration) from an exercise event or can also be an intermediate peak value from an interval session conducted as part of the total exercise event. Lactate clearance rates during recovery can then be compared (e.g., by determining the first derivative of the lactate clearance curve) between the interval sessions to determine a peak lactate clearance rate. The processor within the systems may facilitate determination of the lactate clearance rate.

Accordingly, systems of the present disclosure may comprise a lactate-responsive sensor adapted for detecting lactate in vivo, and a processor located in a cloud server, remote terminal or a local terminal that is communicatively coupled to the lactate-responsive sensor. Cloud- or server-based communication also falls within the scope of the systems disclosed herein. As used herein, the term “local terminal” refers to a user interface that is physically contiguous with a system where the lactate-responsive sensor is located. For example, in some embodiments, the processor may be contiguous with a housing of the lactate-responsive sensor. As used herein, the term “remote terminal” refers to a user interface that is not located in the same physical space where the lactate-responsive sensor is located. The remote terminal and its processor may be communicatively coupled to the lactate-responsive sensor or a network, however. In some embodiments, an individual interfacing with the system may be blinded to the output of the lactate-responsive sensor. In other embodiments, an individual may see the sensor output (e.g., lactate concentrations) in real-time or near real-time, such as on a remote or local viewable display. Remote terminals may include, for example, a dedicated reader device, a dedicated fitness monitoring device (e.g., a FITBIT), a smart phone, or a smart watch.

The processor in the systems of the present disclosure is adapted to receive a signal from the lactate-responsive sensor. The processor may be further adapted to determine a plurality of lactate concentrations upon receipt of the signal and may further determine a rate of lactate clearance upon processing to determine the lactate concentrations. In some embodiments, the rate of lactate clearance may be determined by calculating a curve slope or a half-life obtained from the lactate concentrations measured by the lactate-responsive sensor. In further embodiments, the processor may be adapted to further determine, based upon the lactate concentrations and/or the rate of lactate clearance (e.g., by a curve slope (first derivative) or lactate half-life), a suitable training protocol for conducting a subsequent exercise event in an interval training program. In illustrative embodiments, the processor may be adapted to determine, based upon the rate of lactate clearance, a suitable intensity, duration, and/or timing for a subsequent exercise event and inform an individual wearing the lactate sensor or another party (e.g., a coach, trainer, instructor, or other interested individual) once an appropriate training protocol has been determined. The coach, trainer or the like may then direct an individual wearing the sensor how a subsequent exercise event should be conducted. The processor may further signal an individual wearing the sensor or another interested party when predetermined lactate levels have been reached, such as a specified lactate concentration, a multiple of a baseline lactate concentration, or a fraction of a peak lactate concentration, for example. The output of the processor may be numerical and/or graphical. The notification to the wearer of the lactate-responsive sensor or other interested party may be auditory, tactile (haptic), or any combination thereof.

Additional details concerning illustrative lactate-responsive sensors suitable for incorporation within the systems and methods of the present disclosure are provided hereinafter. It is to be appreciated, however, that lactate-responsive sensors having architectures, configurations, and/or components different than or in addition to those described expressly hereinafter may also be used suitably in some embodiments of the present disclosure. In general, any lactate-responsive sensor that is suitable for in vivo disposition and in vivo interrogation of a biological fluid may be used in the various embodiments of the present disclosure. In particular embodiments, a housing for the lactate-responsive sensor may be adapted to be worn on-body, and at least a portion of the lactate-responsive sensor may protrude from the housing for insertion in vivo. An active sensing region may be located upon at least a portion of the portion of the lactate-responsive sensor protruding from the housing, particularly the portion of the sensor configured for insertion in vivo. The active sensing region may comprise a sensing layer comprising a polymer and a lactate-responsive enzyme, according to various embodiments.

The active sensing region of lactate-responsive sensors of the present disclosure may be disposed in any suitable location in vivo. Suitable locations may include, but are not limited to, intravenous, subcutaneous, or dermal locations. Intravenous sensors have the advantage of analyzing lactate directly in blood, but they are invasive and can sometimes be painful for an individual to wear over an extended period. Subcutaneous and dermal analyte sensors can often be less painful for an individual to wear due to their shallower penetration and can provide sufficient measurement accuracy in many cases. In some embodiments, lactate-responsive sensors suitable for use in the present disclosure may be dermal sensors configured to interrogate dermal fluid of a user. In other embodiments, lactate-responsive sensors suitable for use in the present disclosure may be configured to interrogate interstitial fluid of a user. As used herein, the term “interrogate” refers to the act of measuring a parameter of a sample.

Lactate-responsive sensors of the present disclosure may extend from a housing that is configured for external wear upon the skin of an individual performing a given physical activity. The external location where the lactate-responsive sensor is placed is not considered to be particularly limited and may be dependent upon the type of physical activity being performed. In illustrative embodiments, the lactate-responsive sensor may be placed upon the biceps, triceps, upper back, lower back, chest, buttocks, abdomen, thigh, or calf. In some embodiments, multiple lactate-responsive sensors may be used to monitor a single exercise event, such as to perform as a comparison between lactate concentrations measured at two different external locations. One sensor may be located at the site of active muscle usage (e.g., on the thigh during cycling), and the other sensor may be positioned at a location having minimal active muscle usage during the exercise event (e.g., on the arm during cycling), thereby allowing the rate of lactate diffusion from the blood stream into other interstitial tissues to be determined. If desired, the outputs from one or both sensor locations can also be cross-referenced with blood lactate readings obtained from finger or ear lobe pricks.

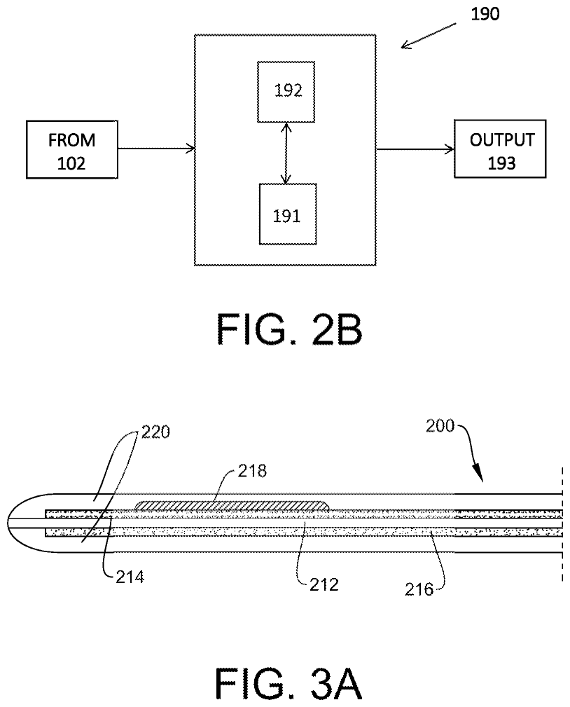

FIG. 2A shows a diagram of an illustrative system that may incorporate a lactate-responsive sensor of the present disclosure. As shown, system 100 includes sensor control device 102 and reader device 120 that are configured to communicate with one another over a local communication path or link, which may be wired or wireless, uni- or bi-directional, and encrypted or non-encrypted. Reader device 120 may constitute an output medium for viewing lactate concentrations and alerts or notifications determined by sensor 104 or a processor associated therewith, as well as allowing for one or more user inputs, according to some embodiments. Alternately, reader device 120 may produce output that is blinded to a user. Reader device 120 may be a multi-purpose smartphone or a dedicated electronic reader instrument. While only one reader device 120 is shown, multiple reader devices 120 may be present in certain instances. A suitable processor may also be incorporated in reader device 120, according to some embodiments. Reader device 120 may also be in communication with remote terminal 170 and/or trusted computer system 180 via communication path(s)/link(s) 141 and/or 142, respectively, which also may be wired or wireless, uni- or bi-directional, and encrypted or non-encrypted. Reader device 120 may also or alternately be in communication with network 150 (e.g., a mobile telephone network, the internet, or a cloud server) via communication path/link 151. Network 150 may be further communicatively coupled to remote terminal 170 via communication path/link 152 and/or trusted computer system 180 via communication path/link 153. Alternately, sensor 104 may communicate directly with remote terminal 170 and/or trusted computer systems 180 without an intervening reader device 120 being present. For example, sensor 104 may communicate with remote terminal 170 and/or trusted computer system 180 through a direct communication link to network 150, according to some embodiments, as described in U.S. Patent Application Publication 2011/0213225 an incorporated herein by reference in its entirety. Any suitable electronic communication protocol may be used for each communication path or link 141, 142, 151, 152 and/or 153, such as near field communication (NFC), radio frequency identification (RFID), BLUETOOTH® or BLUETOOTH® Low Energy protocols, WiFi, mobile telephone network, or the like. Remote terminal 170 and/or trusted computer system 180 may be accessible, according to some embodiments, by a party other than a primary user who have an interest in the primary user's lactate concentrations or rate of lactate clearance, such as the individual's trainer or coach. Reader device 120 may comprise display 122 and optional input component 121. Display 122 may comprise a touch-screen interface, according to some embodiments.

Sensor control device 102 includes sensor housing 103, which may house circuitry and a power source for operating sensor 104. Optionally, the power source and/or active circuitry may be omitted. A processor (not shown in FIG. 2A) may be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

FIG. 2B shows a block diagram of a processing electronics that may be associated with one or more components of the sensing system, such as within reader device 120. Alternately, such functionality may be associated with one or more of network 150, remote terminal 170 or trusted computer system 180. As shown, processing electronics 190 receive, either directly or indirectly, a signal originating from sensor control device 102. The signal may be processed using algorithms associated with processor 191 and/or memory 192. Lactate concentrations determined therewith may be stored in memory 192 and/or exported to output device 193, which may be a display or external storage medium in various embodiments. Guidance, recommendations, and the like may also be determined using processor 191 and exported to output device 193 as well, as described further herein.

Sensor 104 is adapted to be at least partially inserted into a tissue of interest, such as within the dermal layer of the skin or in subcutaneous tissue. Sensor 104 may comprise a sensor tail of sufficient length for insertion to a desired depth in a given tissue. The sensor tail may comprise a sensing region or sensing layer that is active for sensing lactate, and may comprise a lactate-responsive enzyme, according to one or more embodiments. The sensing region or sensing layer may include a polymeric material to which the lactate-responsive enzyme is covalently bonded, according to some embodiments. In various embodiments of the present disclosure, lactate may be monitored in any biological fluid of interest such as dermal fluid, plasma, blood, lymph, synovial fluid, cerebrospinal fluid, saliva, bronchoalveolar lavage, amniotic fluid, or the like. In particular embodiments, lactate-responsive sensors of the present disclosure may be adapted for interrogating dermal fluid or interstitial fluid.

An introducer may be present transiently to promote introduction of sensor 104 into a tissue. In illustrative embodiments, the introducer may comprise a needle. It is to be recognized that other types of introducers, such as sheaths or blades, may be present in alternative embodiments. More specifically, the needle or similar introducer may transiently reside in proximity to sensor 104 prior to insertion and then be withdrawn afterward. While present, the needle or other introducer may facilitate insertion of sensor 104 into a tissue by opening an access pathway for sensor 104 to follow. For example, the needle may facilitate penetration of the epidermis as an access pathway to the dermis to allow implantation of sensor 104 to take place, according to one or more embodiments. After opening the access pathway, the needle or other introducer may be withdrawn so that it does not represent a sharps hazard. In illustrative embodiments, the needle may be solid or hollow, beveled or non-beveled, and/or circular or non-circular in cross-section. In more particular embodiments, the needle may be comparable in cross-sectional diameter and/or tip design to an acupuncture needle, which may have a cross-sectional diameter of about 250 microns, for example. It is to be recognized, however, that suitable needles may have a larger or smaller cross-sectional diameter if needed for particular applications.

In some embodiments, a tip of the needle may be angled over the terminus of sensor 104, such that the needle penetrates a tissue first and opens an access pathway for sensor 104. In other illustrative embodiments, sensor 104 may reside within a lumen or groove of the needle, with the needle similarly opening an access pathway for sensor 104. In either case, the needle is subsequently withdrawn after facilitating insertion.

Sensor 104 may employ a two-electrode or a three-electrode detection motif, according to various embodiments of the present disclosure. Three-electrode motifs may comprise a working electrode, a counter electrode, and a reference electrode. Two-electrode motifs may comprise a working electrode and a second electrode, in which the second electrode functions as both a counter electrode and a reference electrode (i.e., a counter/reference electrode). In both two-electrode and three-electrode detection motifs, the sensing region or sensing layer of sensor 104 may be in contact with the working electrode. In various embodiments, the various electrodes may be at least partially stacked upon one another, as described in further detail hereinafter. In alternative embodiments, the various electrodes may be spaced apart from one another upon the insertion tail of sensor 104.

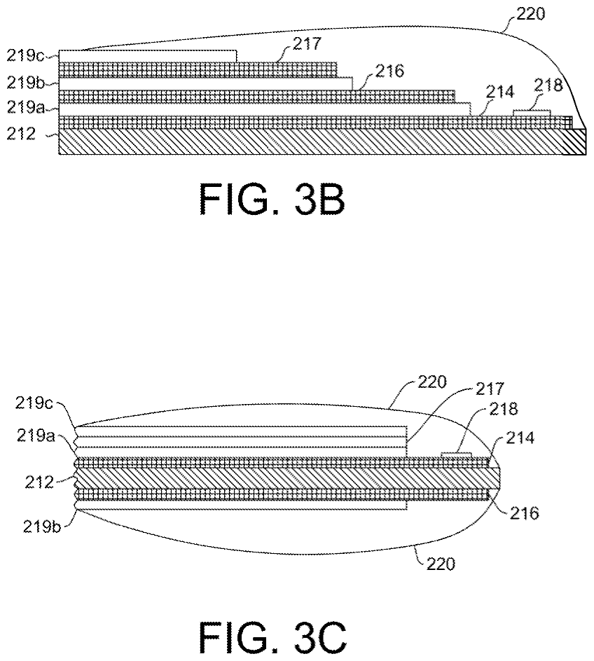

FIG. 3A shows a diagram of an illustrative two-electrode sensor configuration compatible for use in the disclosure herein. As shown, sensor 200 comprises substrate 212 disposed between working electrode 214 and counter/reference electrode 216. Alternately, working electrode 214 and counter/reference electrode 216 may be located upon the same side of substrate 212 with a dielectric material interposed in between. Sensing region 218 is disposed as at least one layer upon at least a portion of working electrode 214. In some embodiments, sensing region 218 may comprise multiple spots or a single spot configured for detection of an analyte of interest, such as lactate. Membrane 220 overcoats at least sensing region 218 and may optionally overcoat some or all of working electrode 214 and/or counter/reference electrode 216, in some embodiments. One or both faces of sensor 200, or the whole of sensor 200, may be overcoated with membrane 220. Membrane 220 may comprise a polymer having capabilities of limiting analyte flux to sensing region 218, specifically the lactate flux in the disclosure herein. Sensor 200 may be operable for interrogating lactate by any of coulometric, amperometric, voltammetric, or potentiometric electrochemical detection techniques.

Three-electrode sensor configurations may be similar to that shown for sensor 200, except for the inclusion of an additional electrode (FIGS. 3B and 3C). With additional electrode 217, counter/reference electrode 216 may then function as either a counter electrode or a reference electrode, and additional electrode 217 (FIGS. 3B and 3C) fulfills the other electrode function not otherwise fulfilled. Working electrode 214 continues to fulfill this function. The additional electrode may be disposed upon either working electrode 214 or counter/reference electrode 216, with a separating layer of dielectric material in between. For example, as depicted in FIG. 3B dielectric layers 219a, 219b and 219c separate electrodes 214, 216 and 217 from one another. Alternately, at least one of electrodes 214, 216 and 217 may be located upon opposite faces of substrate 212 (FIG. 3C). Thus, in some embodiments, electrode 214 (working electrode) and electrode 216 (counter electrode) may be located upon opposite faces of substrate 212, with electrode 217 (reference electrode) being located upon one of electrodes 214 or 216 and spaced apart therefrom with a dielectric material. As with sensor 200 shown in FIG. 3A, sensing region 218 may comprise multiple spots or a single spot configured for detection of an analyte of interest, such as lactate

Additional electrode 217 may be overcoated with membrane 220 in some embodiments. Although FIGS. 3B and 3C have depicted all of electrodes 214, 216 and 217 as being overcoated with membrane 220, it is to be recognized that only working electrode 214 may be overcoated in some embodiments. Moreover, the thickness of membrane 220 at each of electrodes 214, 216 and 217 may be the same or different. As such, the configurations shown in FIGS. 3B and 3C should be understood as being non-limiting of the embodiments disclosed herein. As in two-electrode configurations, one or both faces of sensor 200, or the whole of sensor 200, may be overcoated with membrane 220.

In some embodiments, sensing region 218 may comprise a lactate-responsive enzyme. More particularly, the lactate-responsive enzyme may comprise lactate dehydrogenase or lactate oxidase, according to various embodiments of the present disclosure. In some embodiments, sensing region 218 may further comprise a stabilizer for lactate dehydrogenase or lactate oxidase, such as catalase. According to still more specific embodiments, the lactate-responsive enzyme, such as lactate dehydrogenase or lactate oxidase, may be covalently bonded to a polymer comprising sensing region 218. Covalent bonding immobilizes the lactate-responsive enzyme upon working electrode 214.

In still more specific embodiments, sensing region 218 may comprise a polymer that is covalently bonded to both the lactate-responsive enzyme, such as lactate dehydrogenase or lactate oxidase, and a low-potential osmium complex electron transfer mediator, as disclosed in, for example, U.S. Pat. No. 6,134,461, which is incorporated herein by reference in its entirety. The electron transfer mediator may facilitate conveyance of electrons from lactate to working electrode 214 during a redox reaction. Changes in the signal intensity (e.g., current) at working electrode 214 may be proportional to the lactate concentration and/or the activity of the lactate-responsive enzyme. A calibration factor may be applied (e.g., by a processor) to determine the lactate concentration from the signal intensity, according to some embodiments. Suitable electron transfer mediators include electroreducible and electrooxidizable ions, complexes or molecules having redox potentials that are a few hundred millivolts above or below the redox potential of the standard calomel electrode (SCE). Other suitable electron transfer mediators may comprise metal compounds or complexes of ruthenium, iron (e.g., polyvinylferrocene), or cobalt, for example. Suitable ligands for the metal complexes may include, for example, bidentate or higher denticity ligands such as, for example, a bipyridine, biimidazole, phenanthroline, or pyridyl(imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in the metal complex to achieve a full coordination sphere.

Suitable polymers for inclusion in sensing region 218 include, but are not limited to, polyvinylpyridines (e.g., poly(4-vinylpyridine)), polyimidazoles (e.g., poly(1-vinylimidazole), or any copolymer thereof. Illustrative copolymers that may be suitable include, for example, copolymers containing monomer units such as styrene, acrylamide, methacrylamide, or acrylonitrile.

Covalent bonding of the lactate-responsive enzyme to a polymer or other matrix (e.g., sol-gel) in sensing region 218 may take place via a crosslinker introduced with a suitable crosslinking agent. Suitable crosslinking agents for reaction with free amino groups in the enzyme (e.g., with the free amine in lysine) may include crosslinking agents such as, for example, polyethylene glycol diglycidylether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme may include, for example, carbodiimides.

Although the lactate-responsive enzyme and/or the electron transfer mediator may be covalently bonded to a polymer or other suitable matrix in sensing region 218, other association means may be suitable as well. In some embodiments, the lactate-responsive enzyme and/or the electron transfer mediator may be ionically or coordinatively associated with the polymer or other matrix. For example, a charged polymer may be ionically associated with an oppositely charged lactate-responsive enzyme or electron transfer mediator. In still other embodiments, the lactate-responsive enzyme and/or the electron transfer mediator may be physically entrained within the polymer or other matrix of sensing region 218.

In alternative embodiments, one or more components of sensing region 218 may be solvated, dispersed, or suspended in a fluid, instead of being disposed in a solid composition. The fluid may be provided with sensor 200 or may be absorbed by sensor 200 from the biological fluid that is undergoing analysis. In some embodiments, the components which are solvated, dispersed, or suspended in this type of sensing region 218 are non-leachable from sensing region 218. In some embodiments, non-leachability may be accomplished, for example, by providing barriers (e.g., membranes and/or films) around sensing region 218. One example of such a barrier is a microporous membrane or film, which allows diffusion of lactate into sensing region 218, but reduces or eliminates diffusion of sensing region 218 components (e.g., an electron transfer agent, an enzyme and/or a reactant) out of sensing region 218. Such barriers may, in some embodiments, be considered as flux-limiting membranes and may avoid saturating sensor 200 when excessive lactate is present. Flux-limiting membranes of this type may also be used when sensing region contains primarily solid components, as referenced above.

Sensor 200 may also be configured to analyze for other analytes as well. For example, according to some embodiments, sensor 200 may be further adapted for detecting glucose in vivo by also incorporating suitable sensing functionality for this analyte. Additional analytes that may be of interest in the sports training realm include, for example, markers of cardiac stress, markers of inflammation, pyruvate, pH, triglycerides, free fatty acids, and hormones such as insulin, glucagon, cortisol, epinephrine, norepinephrine, testosterone, HGH, IFG1, and BDNF.

In still other embodiments, system 100 may incorporate further functionality appropriate for monitoring physical activity. Additional functionality that may optionally be present include, for example, a heart rate monitor, a blood oxygen monitor, a power meter, an accelerometer, a pedometer, or the like.

It is to be appreciated that system 100 and sensor 200 may comprise additional features and/or functionality that are not necessarily described herein in the interest of brevity. Thus, the foregoing description of analyte monitoring system 100 and sensor 200 should be considered illustrative and non-limiting in nature.

Methods for monitoring lactate levels during a fitness training activity may employ one or more configurations of the lactate-responsive sensors described hereinabove. In various embodiments, the methods for monitoring lactate levels according to the present disclosure may comprise determining a plurality of lactate concentrations as the lactate concentrations are decreasing after reaching a peak lactate level (concentration). As such, the methods described herein utilize a lactate-responsive sensor to analyze lactate levels dynamically in order to determine lactate clearance or the rate thereof. In some embodiments, the rate of lactate clearance or time to peak lactate may be utilized as a metric of physical fitness (i.e., by correlating the time to peak lactate or the rate of lactate clearance or lactate levels to one's physical fitness) and/or to suggest a training protocol for a subsequent physical activity (exercise event). In other embodiments, lactate concentrations that are a fixed number, a percentage of a peak lactate level, or a multiple of a baseline lactate concentration may be used to dictate future training protocols.

Certain methods of the present disclosure may comprise: interrogating a biological fluid of an individual in vivo with a lactate-responsive sensor following a first exercise event in which a lactate level above a baseline concentration has been reached; measuring a plurality of lactate concentrations in the biological fluid with the lactate-responsive sensor over at least a period of time while the lactate concentrations are decreasing following a peak lactate level reached in conjunction with the first exercise event; and directing or conducting a second exercise event after a recovery period in which the lactate level has fallen to a predetermined concentration. The recovery period intercedes between the first exercise event and the second exercise event. The peak lactate level may occur during the first exercise event or following the first exercise event. An individual wearing the lactate-responsive sensor may make a decision about how the second exercise event is to be conducted, or the individual make take direction from a coach, trainer, or similar person.

Other related methods of the present disclosure may comprise: measuring a plurality of lactate concentrations in a biological fluid in vivo with a sensing system comprising a lactate-responsive sensor over at least a period of time while the lactate concentrations are decreasing following a peak lactate level reached in conjunction with a first exercise event, a lactate level above a baseline concentration being reached in the first exercise event; and specifying, based on an output of the sensing system, that a second exercise event be conducted after a recovery period in which the lactate level has fallen to a predetermined concentration, the recovery period interceding between the first exercise event and the second exercise event.

According to various embodiments, the first exercise event and the second exercise event may represent at least a portion of an interval training protocol. In some embodiments, the first exercise event and the second exercise event may be the same type of physical activity. In interval training, for example, the first exercise event and the second exercise event are commonly the same. Interval training has conventionally been conducted in this manner. In other embodiments, however, the first exercise event and the second exercise event may be different. Current paradigms in interval training also encompass situations wherein the first exercise event and the second exercise event are different types of exercise or are performed differently. For example, in some embodiments, the first exercise event may be an anaerobic activity and the second exercise event may be an aerobic activity.

Suitable types of exercise events that may be practiced during interval training and in the other various embodiments disclosed herein are not considered to be especially limited. Depending on particular training needs, suitable exercise events may be conducted aerobically or anaerobically. Aerobic activities do not produce a substantial amount of lactate. Anaerobic activities, in contrast, produce lactate through glycolysis during intense physical activity. Moreover, a second (subsequent) exercise event need not necessarily be conducted with the same intensity, duration or rest period associated with a first (initial) exercise event. Suitable exercise events that may be associated with the various methods of the present disclosure include, for example, running, walking, cycling, rowing, weight lifting, circuit training, alpine skiing, cross-country skiing, skating, rowing, and the like. Hybrid (combination) exercise events may also be suitable in the embodiments described herein. Other sports that may benefit from the training methods of the present disclosure include, for example, baseball, softball, basketball, football, lacrosse, martial arts, wrestling, tennis, golf, volleyball, racquetball, squash, surfing, snowboarding, rock climbing, mountain climbing, hiking, gymnastics, and track and field sports.

Baseline concentrations of lactate may vary from individual to individual. Accordingly, a determination of the baseline concentration of lactate may be made in the various methods disclosed herein. The baseline concentration of lactate may, in some embodiments, facilitate a determination of when lactate levels have fallen sufficiently to conduct a second exercise event (e.g., in an interval training protocol). The baseline concentration of lactate may be determined prior to reaching lactate threshold and/or after the lactate concentrations have again stabilized after reaching a lactate threshold. The lactate-responsive sensor may be used to determine the baseline concentration, or the baseline concentration may be determined through laboratory-based measurement of lactate. In illustrative embodiments, the baseline lactate concentration may range between about 0.5 mM and about 1.0 mM.

According to the various embodiments described herein, a plurality of lactate concentrations may be measured in a biological fluid while the lactate concentrations are decreasing following a peak lactate level being reached. The peak lactate level may be below, above, or at the lactate threshold of a given exercise event for a particular individual. In various individuals, peak lactate levels may vary over a broad range. For endurance athletes (little anaerobic contribution), peak lactate levels may range up to about 6 mM. For glycolytic/anaerobic athletes, peak lactate levels up to about 20-25 mM are possible. The lactate threshold may occur over a range of about 3 mM to about 7 mM, for example, depending on the individual. Regardless of whether the lactate threshold is exceeded or not during a given exercise event and for a given type of exercise, monitoring lactate clearance according to the present disclosure may be advantageous and allow a determination of an individual's physical fitness to be made.

After a first exercise event (interval) has been conducted, a recovery period may follow before a second exercise event takes place. The length of the recovery period and activities conducted during the recovery period may be dictated by the rate of lactate clearance, which can be determined according to the disclosure herein. Before directing or conducting a second exercise event, the lactate level is allowed to fall to a predetermined concentration in the recovery period. In various embodiments, the predetermined concentration is at or above the baseline concentration. In some cases, allowing the lactate level to fall all the way to the baseline concentration may be desirable before conducting the second exercise event or directing that the second exercise event be conducted. For example, it may be desirable for the baseline concentration of lactate to be reached before participating in a competition, whereas during training it may be desirable not to reach the baseline concentration. In other instances, a second exercise event may achieve a desired training goal when the second exercise event is conducted with the lactate level above the baseline concentration. As such, in some embodiments, the predetermined concentration may be a set number, such as a multiple of the baseline concentration, or a variable number, such as a percentage of the peak lactate level. In general, the more recovery time allotted to an individual, the more intense the second exercise event can be. In illustrative embodiments, the second exercise event may be conducted after a sufficient recovery time such that the second exercise event is not impacted negatively (e.g., failure to reach a desired intensity or peak heart rate). In other illustrative embodiments, a second exercise event may be conducted after lactate levels have fallen only minimally in order to increase tolerance to lactate loading during exercise.

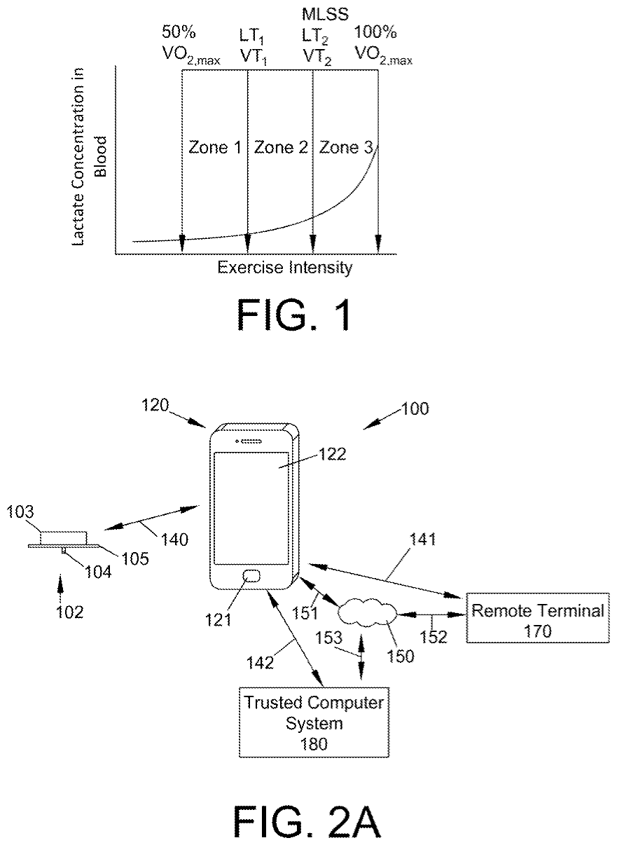

Depending on an individual's desired training goals, the recovery period following a given exercise event may be managed in various ways. In some embodiments, the recovery period may be made longer or shorter than planned in response to an observed rate of lactate clearance. In other embodiments, activities within the recovery period may alter the rate of lactate clearance as required to meet a specified training goal. In some embodiments, the rate of lactate clearance may occur at its native rate, with no exercise being conducted during the recovery period. In other embodiments, moderate exercise or a reduced level (intensity) of exercise may be conducted during the recovery period. For example, moderate exercise following an intense anaerobic activity may increase the rate of lactate clearance over that otherwise occurring at its native rate. Depending on the type of exercise being conducted, how much lactate accumulates, and the intensity at which the moderate exercise in the recovery period is conducted, the rate of lactate clearance in the recovery period may be increased or decreased as desired to meet a specified training goal. According to some embodiments, moderate exercise may constitute that occurring within Zone II (see FIG. 1) where the participant has progressed from below LT1/VT1 (Zone 1) and demonstrates increasing lactate concentrations. Zone 2 is bounded by LT1/VT1 and LT2/VT2, wherein LT1 is the aerobic threshold and LT2 is the anaerobic threshold and VT1 and VT2 are the first and second ventilator threshold respectively. VT1 is where ventilation starts to increase in a non-linear fashion and VT2 is the point which high-intensity exercise can be no longer be sustained due to an accumulation of lactate. VO_{2,max} is the maximum rate of oxygen consumption measured during incremental exercise and expressed as an absolute rate in liters of oxygen per min (L/min) or relative rate per kilogram of body mass ((mL/kg-min)). Maximal Lactate Steady State (MLSS) is defined as the highest blood lactate concentration that can be maintained over time without continual blood lactate accumulation. In another example, moderate exercise may be representative of an exercise intensity below that needed to reach the lactate threshold and above the initial rise of lactate (i.e., between Zones I and II).

Methods of the present disclosure may comprise determining a rate of lactate clearance in the recovery period from the plurality of lactate concentrations. Depending upon whether the rate of lactate clearance is linear or non-linear, the determined rate may vary at different points in time in the recovery period. Determining the rate of lactate clearance may comprise calculating a curve slope (i.e., the first derivative) or a half-life of the lactate concentrations measured in the recovery period. The lactate clearance curve may show zero-, first-, and/or second-order kinetics. Clearance is variable in zero-order kinetics as a constant amount of lactate is eliminated per unit time and is not dependent upon the remaining lactate concentration. In first-order kinetics, the amount of lactate cleared is dependent upon the remaining lactate concentration so that a constant fraction is eliminated per unit time, thereby allowing calculation of a rate constant. The majority of the lactate clearance curve may provide first-order kinetics to yield a first derivative rate constant. It is to be appreciated that the curve slope or half-life may be calculated without obtaining an actual physical or visual plot of the lactate concentration data. Thus, according to various embodiments, the lactate concentration data may be computationally processed in order to calculate a curve slope or a half-life associated with the rate of lactate clearance. The rate of lactate clearance may be utilized to forward project the lactate level of an individual at a future point in time, according to various embodiments. Thus, if lactate levels following a first exercise event have not yet fallen sufficiently to conduct a second exercise event with a desired training goal in mind, the rate of lactate clearance may be used to project when the second exercise event may be conducted. Alternately, the rate of lactate clearance itself or a change thereof may be representative of an individual's physical fitness.

Measured lactate levels result from the dynamic equilibrium of lactate production in the working muscles, oxidation and reconversion within muscle cells and surrounding muscle fibers, diffusion into the blood, and removal from the blood through buffering, excretion, and uptake for oxidation and reconversion in other tissues (e.g., in the heart and liver). These actions represent the balance between the rate of lactate production and the rate of lactate disposal or clearance. As measured lactate levels represent a balance between two variables, assessing the relative contributions from each variable is difficult. One potential mechanism for assessing the disposal side of the balance is to measure the rate of lactate clearance post exercise. Because the lactate clearance rate represents the body's ability to oxidize and reconvert lactate as energy, it may be indicative of aerobic fitness and may be used to establish training interventions. Monitoring changes in lactate clearance can likewise be used to assess the efficacy of the training interventions.

Increases in performance have been shown to be primarily linked to increasing the rate of lactate disposal. During exercise, active muscles account for the majority of lactate disposal. Improved lactate clearance rates following exertion, but at a reduced workload below the lactate threshold, is a direct reflection of the muscles' ability to oxidize lactate. This capacity is a result of enhancements to the muscles' aerobic fitness, such as through increased mitochondrial density and associated proteins.

According to further embodiments of the present disclosure, at least one of intensity (as measured by power, percentage of VO₂ max, heart rate, acceleration, speed, or the like), duration (i.e., the length of time an exercise event is conducted) and timing (i.e., when an exercise event is conducted, particularly the timing for conducting a second exercise event relative to a first exercise event) may be adjusted in response to the rate of lactate clearance. Adjustment of one or more of intensity, duration, or timing of the second (subsequent) exercise event may promote a desired training outcome. The intensity, duration, or timing of the second exercise event may be chosen in response to, for example, how far the lactate concentrations have fallen from the peak lactate level, how close the lactate concentrations are to the baseline level, and/or how long it has taken to reach a predetermined lactate concentration. The manner in which the second (subsequent) exercise event is conducted may occur with an increased level of physical fitness in mind. For example, the second (subsequent) exercise event may be conducted with the intent of one or more of increasing the rate of lactate clearance, increasing an individual's lactate threshold, increasing the exercise intensity or duration before the onset of the lactate threshold, increasing an individual's peak lactate level achieved before an activity must be stopped, or any combination thereof. Fat-burning, increasing muscle mass, increasing speed, and/or increasing endurance may also be outcomes that are sought by the manner in which the second exercise event is conducted. Accordingly, certain methods of the present disclosure may further comprise: determining a rate of lactate clearance in the recovery period using the sensing system and the plurality of lactate concentrations; and specifying, based on the output of the sensing system and the rate of lactate clearance, at least one of intensity, duration, or timing of the second exercise event.

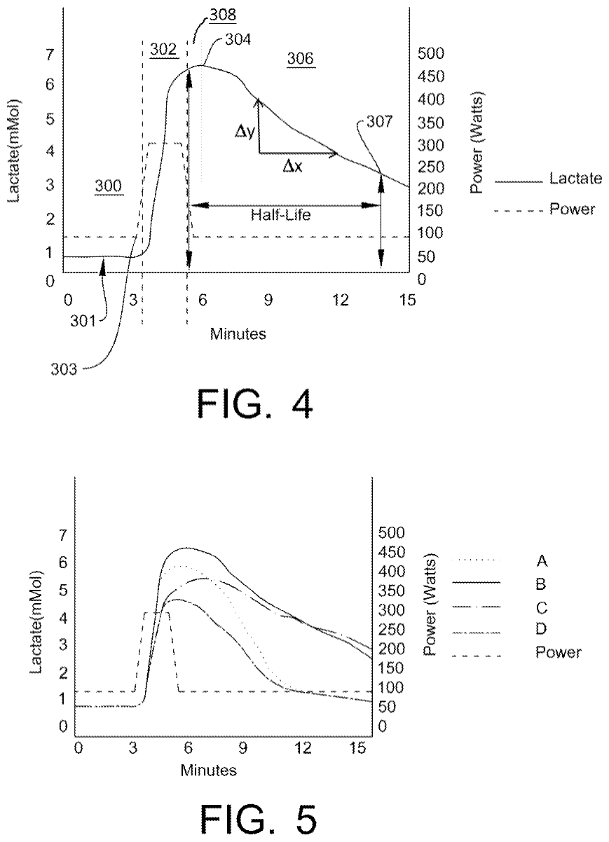

FIG. 4 shows an illustrative plot of lactate concentration as a function of time for a single exercise event. As shown, the rates of lactate production and lactate clearance are largely in balance with one another in region 300, prior to the onset of (intense) exercise, and the lactate concentrations remain steady or increase only marginally above baseline concentration 301. The onset and cessation of exercise is indicated by the dashed power line in FIG. 4. Balanced production and clearance of lactate is characteristic of lower intensity or more tolerated physical activity. In region 302, however, the rate of lactate production exceeds the rate of lactate clearance, and lactate concentrations rapidly rise with the onset and continuation of intense exercise, until peak lactate level 304 is reached and the physical activity is stopped by design at the direction of a coach or trainer or due to exhaustion. Lactate threshold 303 represents the intensity of exertion (physical activity) needed to transition from region 300 into region 302. After reaching peak lactate level 304, either during an exercise event or after completing an exercise event, lactate concentrations begin to fall in region 306, which represents a recovery period for the exercise event and the beginning of lactate clearance. During the recovery period in region 306, no exercise (i.e., no intensity) or reduced intensity exercise (i.e., below the intensity needed to reach lactate threshold 303) is conducted, with the intensity during the recovery period being chosen to impact the rate of lactate clearance in a desired way (i.e., leave the rate of lactate clearance unchanged or increase/decrease the rate of lactate clearance). Moderate exercise during recovery may speed up lactate clearance, for example. In region 306, lactate concentrations may be allowed to fall until predetermined level 307 is reached. In the illustrated embodiment, the predetermined level 307 is half that of peak lactate level 304. As discussed above, predetermined level 307 may represent baseline concentration 301, a set number, a percentage of peak lactate level 304, or a multiple of baseline concentration 301.

Peak lactate level 304 may often occur in region 306 after the cessation of intense physical activity, with Time to Peak 308 being defined as the length of time elapsed from the cessation of exercise until the lactate reaches a peak value, as shown in FIG. 4. Alternately, peak lactate level 304 may occur in region 302, in particular at a sub-threshold intensity level, while an exercise event is still ongoing. For example, sensing systems of the present disclosure or a trainer may direct that a physical activity be stopped at a sub-maximal lactate concentration in order to meet a specified training goal. Specified training goals may include, but are not limited to, fat-burning, increasing muscle mass, increasing speed, and/or increasing endurance.

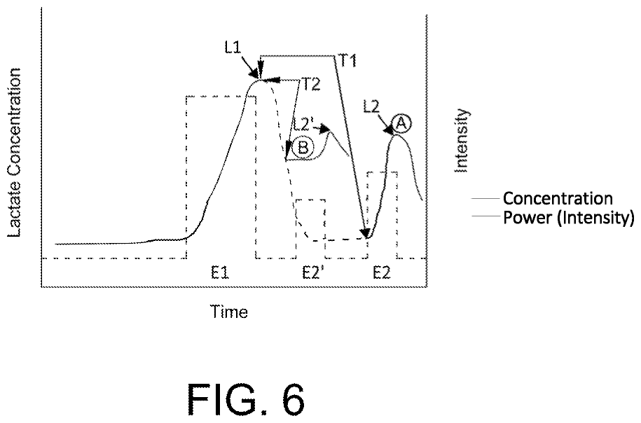

Various individuals may exhibit differing baseline concentrations of lactate, peak lactate levels, time to reach peak lactate levels, and rates of lactate clearance. FIG. 5 shows an illustrative plot demonstrating how these parameters may vary between different individuals in a single exercise event and how the differences may be representative of one's physical fitness. As shown, individuals A-D may have different lactate clearance profiles even when conducting a physical activity with approximately the same intensity. Individual B has the highest peak lactate level and a fairly slow rate of lactate clearance. Individual C has a lower peak lactate level but a rate of lactate clearance that is approximately equal to that of individual B. Individuals A and D reach their peak lactate levels earlier and achieve lactate clearance faster than do individuals B and C, as indicated by the greater negative curve slope. Thus, individuals B and C take much longer than individuals A and D to recover from a similar level of physical activity. By this metric, individuals A and D may be considered more physically fit than individuals B and C. Individual A exhibits the highest peak lactate concentration and a greater rate of lactate clearance. Thus, by these metrics, individual A may be considered the most physically fit. Comparing peak lactate levels, in contrast, leads to the conclusion that individual B is the most physically fit, in spite of their relatively low rate of lactate clearance. Depending upon an individual's particular training goals, any of the above metrics, or others, may represent an acceptable measure for determining physical fitness. The more rapid lactate clearance of individuals A and D may be indicative of their having performed moderate exercise during recovery, for example, to facilitate clearance of lactate from bodily tissues. The moderate exercise is not shown in the power line in FIG. 5. In other aspects, the more rapid lactate clearance of individuals A and D may be indicative of their being more physically fit than are individuals B and C at the time the exercise event was being conducted.

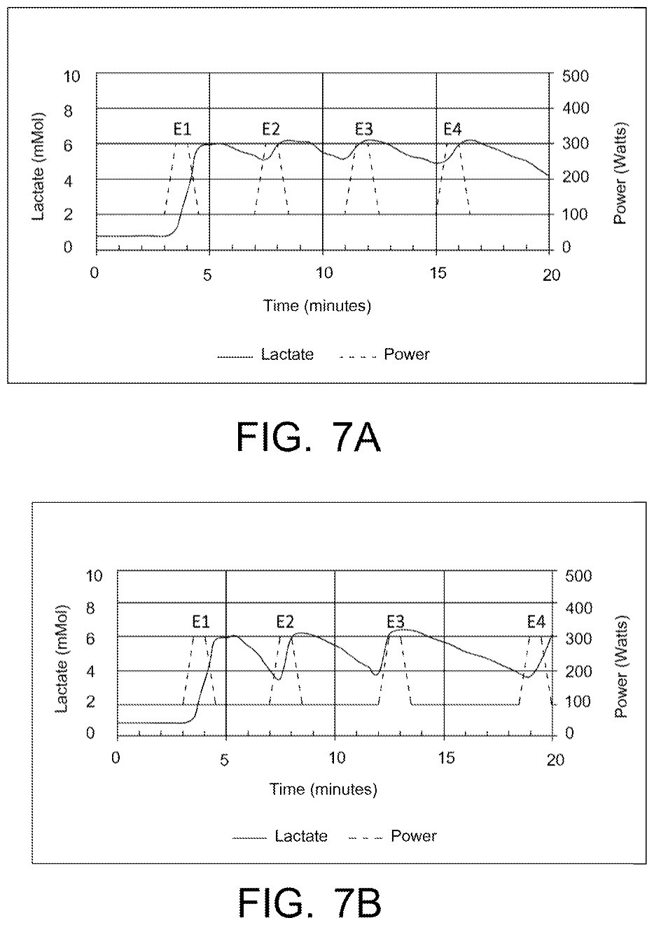

According to various embodiments of the present disclosure, the rate of lactate clearance following a first exercise event may be analyzed by the sensing systems to adjust at least one of intensity, duration, or timing of a second exercise event. FIG. 6 shows an illustrative plot showing how parameters of a second exercise event may be adjusted in response to the rate of lactate clearance following a first exercise event. As discussed above, adjusting at least one of intensity, duration, or timing of a second exercise event may allow a specified training goal to be met. In FIG. 6, peak lactate concentration L1 is reached during the first exercise event. After L1 is reached, no exercise is conducted (dashed portion of the lactate concentration line) until a specified later time, which may be prompted by the sensing systems according to one or more embodiments. In one instance (curve A in FIG. 6), for example, lactate concentrations may be allowed to fall to or near the baseline concentration of lactate following a first exercise event (E1), thereby increasing the length of the recovery period to time interval T1 before a second exercise event (E2) takes place, in which lactate concentration L2 is reached. In another instance (curve B in FIG. 6), a second exercise event (E2′) may be conducted after a much shorter recovery period at the end of time interval T2 before lactate concentrations have fully returned to the baseline concentration, while again attaining lactate concentration L2′. When second exercise event E2′ is performed as shown in Curve B, second exercise event E2′ may or may not be performed. In Curve B, the lactate levels were approximately 50% of peak and the second exercise event (E2) was about 30% effort of the first exercise event. Although the second exercise events depicted in both curves A and B in FIG. 6 have been shown as reaching approximately the same peak lactate level (L2=L2′), it is to be recognized that the chosen peak lactate level may vary based upon desired training goals. The intensity at which the second exercise event is conducted may be chosen based upon how long the recovery period has been and how much it is desired for the lactate levels to rise during the second exercise event. For curve A, the lactate level was allowed to recover near baseline, whereupon the individual could perform a harder second exercise event (E2) compared to E2′ in Curve B. Continuous lactate monitoring permits contemporaneous time and intensity adjustments of successive exercise efforts to take place. Such efforts may be considerably facilitated by monitoring lactate concentrations and receiving prompts from the sensing systems disclosed herein.

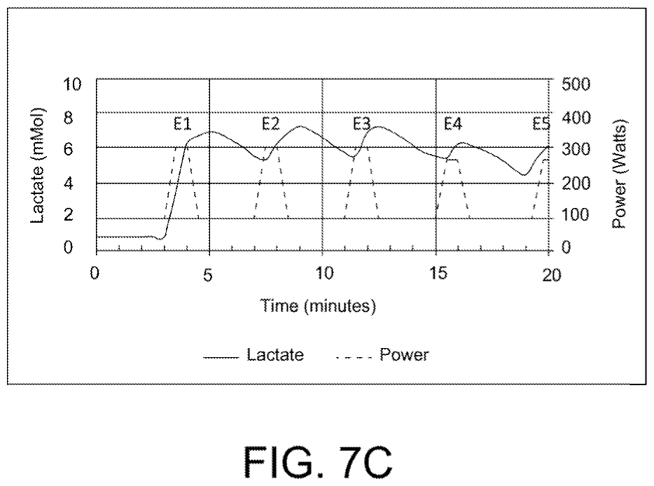

FIGS. 7A-7C show illustrative plots demonstrating how lactate levels may respond to altering the intensity, duration, or timing of subsequent exercise events in a plurality of exercise events. FIG. 7A shows a plurality of intervals (E1-E4) in which the recovery period and peak lactate levels are approximately the same in each interval. FIG. 7B, in contrast, shows an adaptive recovery process, in which later intervals (after E3) are performed with a longer recovery period to achieve sufficient lactate clearance and/or to moderate peak lactate levels. FIG. 7C, in still further contrast, shows an adaptive power process, in which later intervals (E4 and E5) are performed at a lower intensity than are earlier intervals in order to moderate peak lactate levels and/or lactate clearance rates across multiple intervals. An intra-workout modulation of peak lactate levels and clearance rates may promote more efficient interval training, according to some embodiments.

In addition to interrogating a biological fluid to measure lactate levels while the lactate concentrations are decreasing, some embodiments of the present disclosure may feature the plurality of lactate concentrations including one or more lactate concentrations measured prior to reaching a peak lactate level (i.e., while the lactate concentrations remain steady or are increasing). By measuring the second plurality of lactate concentrations, one may determine when the lactate threshold or the peak lactate concentration has been reached. At least a portion of the plurality of lactate concentrations measured prior to the peak lactate concentration may be representative of the baseline concentration, according to some embodiments. In various embodiments, the lactate concentrations may be monitored continuously over the course of an interval training session with the lactate-responsive sensor in order to realize the benefits described herein. Thus, it is to be appreciated that the various embodiments of the present disclosure are not limited to only those in which lactate concentrations are measured in the recovery period. According to various embodiments, a single lactate-responsive sensor may interrogate a biological fluid over a given period of time, either continuously throughout a particular training period or as needed during a recovery period.

Although some of the preceding description has shown how the rate of lactate clearance following a first exercise event may be analyzed for modifying the way a second exercise event is conducted, it is to be appreciated that the principles of the present disclosure may also be applicable to interval training protocols in which more than two exercise events are conducted. Again, depending upon an individual's desired training goals, each exercise event (activity) in a plurality of exercise events may be of the same type or different. Moreover, according to various embodiments of the present disclosure, one or more of intensity, timing, or duration of a subsequent exercise event may be prompted or specified for adjustment in response to the rate of lactate clearance following a prior exercise event. Alternately, one or more of the intensity, timing, or duration of the recovery period following an exercise event may be prompted or specified for adjustment in response to the rate of lactate clearance following a prior exercise event.

According to some specific embodiments, methods for interval training may comprise: directing or performing interval training in which a plurality of exercise events are conducted over a period of time; wherein a lactate level above a baseline concentration is reached in or following each exercise event; measuring a plurality of lactate concentrations in a biological fluid of an individual performing the interval training, the plurality of lactate concentrations being measured in vivo with a lactate-responsive sensor; wherein the plurality of lactate concentrations are measured at least while the lactate concentrations are decreasing in a recovery period following a peak lactate level reached in conjunction with each exercise event; calculating a rate of lactate clearance during the recovery period following each exercise event using the lactate concentrations; and determining, based upon the rate of lactate clearance, at least one of intensity, duration, or timing of a subsequent exercise event or a recovery (rest) period to meet a specified training goal. According to various embodiments, the subsequent exercise event may be conducted at a different intensity or duration than was an initial exercise event in which the peak lactate level was reached. The peak lactate level may be reached during the exercise event or following the exercise event.

Other related methods for performing interval training according to various embodiments of the present disclosure may comprise: measuring a plurality of lactate concentrations in a biological fluid of an individual performing interval training in which a plurality of exercise events are conducted over a period of time and a lactate level above a baseline concentration is reached in each exercise event, the plurality of lactate concentrations being measured in vivo with a sensing system comprising a lactate-responsive sensor; wherein the plurality of lactate concentrations are measured at least while the lactate concentrations are decreasing in a recovery period following a peak lactate level reached in conjunction with each exercise event; calculating a rate of lactate clearance during the recovery period following each exercise event using the sensing system and the lactate concentrations measured therewith; and specifying, based on an output of the sensing system, that at least one of intensity, duration, or timing of a subsequent exercise event be modified to meet a specified training goal.

Some or other embodiments of methods for performing interval training may comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining when a second exercise event should take place to meet a specified training goal based on the rate of lactate clearance. Specified training goals may include, but are not limited to, fat-burning, increasing muscle mass, increasing speed, and/or increasing endurance. The peak lactate level may be reached during the exercise event or following the exercise event. According to some embodiments, the methods may further comprise adjusting intensity or duration of the second exercise event or a recovery period in response to the rate of lactate clearance.

Other related methods for performing interval training according to various embodiments of the present disclosure may comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, when a second exercise event should take place to meet a specified training goal.

Some or other embodiments of methods for performing interval training may comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining an intensity at which a second exercise event should take place to meet a specified training goal based on the rate of lactate clearance. Specified training goals may include, but are not limited to, fat-burning, increasing muscle mass, increasing speed, and/or increasing endurance. The peak lactate level may be reached during the exercise event or following the exercise event. According to some embodiments, the methods may further comprise adjusting timing or duration of the second exercise event in response to the rate of lactate clearance. According to some or other embodiments, the methods may further comprise adjusting one or more of the intensity, timing and/or duration of the recovery period in response to the rate of lactate clearance.

Other related methods for performing interval training according to various embodiments of the present disclosure may comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, an intensity at which a second exercise event should take place to meet a specified training goal.

Some or other embodiments of methods for performing interval training may comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining how long a second exercise event should take place (i.e., duration) to meet a specified training goal based on the rate of lactate clearance. Specified training goals may include, but are not limited to, fat-burning, increasing muscle mass, increasing speed, and/or increasing endurance. The peak lactate level may be reached during the exercise event or following the exercise event. According to some embodiments, the methods may further comprise adjusting intensity or timing of the second exercise event in response to the rate of lactate clearance.

Other related methods for performing interval training according to various embodiments of the present disclosure may comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, how long a second exercise event should take place to meet a specified training goal.

In some embodiments, the foregoing methods for performing interval training may be conducted such that the peak lactate level is above a lactate threshold and the subsequent exercise event is conducted after the lactate level has fallen below the lactate threshold. In some embodiments, the subsequent exercise event is conducted after the lactate level has fallen to the baseline concentration. In other embodiments, the subsequent exercise event may be conducted after the lactate level has fallen to a set number, a percentage of the peak lactate level, or a multiple of the baseline concentration. The manner in which the subsequent exercise event is conducted may be selected to meet particular training goals, as discussed above. Guidance of how to perform the subsequent exercise event may be provided based upon an output of the sensing systems and/or at the behest of a trainer who may make decisions on how to achieve one or more particular training goals based on a currently measured lactate concentration.

Other interval training embodiments may be conducted without the lactate threshold being exceeded. In more specific embodiments, the peak lactate level may be a lactate threshold, and the subsequent exercise event is conducted after the lactate level has fallen to a predetermined concentration above the baseline concentration. In some embodiments, the subsequent exercise event is conducted after the lactate level has fallen to the baseline concentration. In other embodiments, the subsequent exercise event may be conducted after the lactate level has fallen to a set number, a percentage of the peak lactate level, or a multiple of the baseline concentration.

According to some embodiments, the lactate threshold or the rate of lactate clearance may change over time, specifically between one or more of the exercise events in the interval training. The change in the lactate threshold or the rate of lactate clearance may be diagnostic of an individual's physical fitness.

In some embodiments, the foregoing methods for performing interval training may comprise adjusting or regulating the rate of lactate clearance in the recovery period. In more specific embodiments, the rate of lactate clearance may be adjusted as desired by conducting no exercise or a reduced level of exercise during the recovery period. Still more specific embodiments may include those in which an output of the sensing systems specify that no exercise or a reduced level of exercise be performed during the recovery period in order to adjust the rate of lactate clearance.

In the interval training methods described herein, a processor may receive a signal from a lactate-responsive sensor in order to determine a plurality of lactate concentrations. The processor may further determine, based upon the plurality of lactate concentrations, the rate of lactate clearance and suggest or specify a training protocol for conducting a subsequent (second) exercise event based upon the calculated rate of lactate clearance. In more specific embodiments, the interval training methods may comprise communicating a signal from the lactate-responsive sensor to a processor located in a remote terminal, a local terminal, or a cloud server. The processor may determine, based upon the plurality of lactate concentrations, the rate of lactate clearance and a training protocol including intensity of a second exercise event, duration of a second exercise event, timing of a second exercise event, and combinations thereof. The processor may further inform an individual performing the interval training or another party directing the interval training once the training protocol is available.

Some or other more specific embodiments of methods for performing interval training may comprise: directing or performing interval training in which a plurality of exercise events are conducted over a period of time; wherein at least a portion of the exercise events are conducted at an intensity such that a lactate threshold is exceeded; measuring a plurality of lactate concentrations in a biological fluid of an individual performing the interval training, the plurality of lactate concentrations being measured in vivo with a lactate-responsive sensor; wherein the plurality of lactate concentrations are measured at least while the lactate concentrations are decreasing in a recovery period following a peak lactate level reached during each exercise event; communicating a signal from the lactate-responsive sensor to a processor located in a remote terminal or a local terminal; calculating, with the processor, a rate of lactate clearance during the recovery period following each exercise event, the rate of lactate clearance being based upon the plurality of lactate concentrations; and determining with the processor, based upon the rate of lactate clearance, at least one of intensity, duration, or timing of a subsequent exercise event to meet a specified training goal. In still more specific embodiments, the processor may determine the plurality of lactate concentrations upon receipt of the signal.

Other related embodiments for performing interval training according to the present disclosure may comprise: measuring a plurality of lactate concentrations in a biological fluid of an individual performing interval training in which a plurality of exercise events are performed over a period of time and at least a portion of the exercise events are conducted at an intensity such that a lactate threshold is exceeded, the plurality of lactate concentrations being measured in vivo with a sensing system comprising a lactate-responsive sensor; wherein the plurality of lactate concentrations are measured at least while the lactate concentrations are decreasing in a recovery period following a peak lactate level reached in conjunction with each exercise event; communicating a signal from the lactate-responsive sensor to a processor located in a remote terminal, a local terminal, or a cloud server; calculating, with the processor, a rate of lactate clearance during the recovery period following each exercise event, the rate of lactate clearance being calculated using the plurality of lactate concentrations; and determining, with the processor and based upon the rate of lactate clearance, that at least one of intensity, duration, or timing of a subsequent exercise event be modified to meet a specified training goal.

Embodiments disclosed herein include:

A. Methods for optimizing exercise. The methods comprise: interrogating a biological fluid of an individual in vivo with a lactate-responsive sensor following a first exercise event in which a lactate level above a baseline concentration has been reached; measuring a plurality of lactate concentrations in the biological fluid with the lactate-responsive sensor over at least a period of time while the lactate concentrations are decreasing following a peak lactate level reached in conjunction with the first exercise event; and directing or conducting a second exercise event after a recovery period in which the lactate level has fallen to a predetermined concentration, the recovery period interceding between the first exercise event and the second exercise event.

A1. Methods for optimizing exercise. The methods comprise: measuring a plurality of lactate concentrations in a biological fluid in vivo with a sensing system comprising a lactate-responsive sensor over at least a period of time while the lactate concentrations are decreasing following a peak lactate level reached in conjunction with a first exercise event, a lactate level above a baseline concentration being reached in the first exercise event; and specifying, based on an output of the sensing system, that a second exercise event be conducted after a recovery period in which the lactate level has fallen to a predetermined concentration, the recovery period interceding between the first exercise event and the second exercise event.

B. Methods for conducting or performing interval training. The methods comprise: directing or performing interval training in which a plurality of exercise events are conducted over a period of time; wherein a lactate level above a baseline concentration is reached in each exercise event; measuring a plurality of lactate concentrations in a biological fluid of an individual performing the interval training, the plurality of lactate concentrations being measured in vivo with a lactate-responsive sensor; wherein the plurality of lactate concentrations are measured at least while the lactate concentrations are decreasing in a recovery period following a peak lactate level reached in conjunction with each exercise event; calculating a rate of lactate clearance during the recovery period following each exercise event using the lactate concentrations; and determining, based upon the rate of lactate clearance, at least one of intensity, duration, or timing of a subsequent exercise event to meet a specified training goal.

B1. Methods for conducting or performing interval training. The methods comprise: measuring a plurality of lactate concentrations in a biological fluid of an individual performing interval training in which a plurality of exercise events are conducted over a period of time and a lactate level above a baseline concentration is reached in each exercise event, the plurality of lactate concentrations being measured in vivo with a sensing system comprising a lactate-responsive sensor; wherein the plurality of lactate concentrations are measured at least while the lactate concentrations are decreasing in a recovery period following a peak lactate level reached in conjunction with each exercise event; calculating a rate of lactate clearance during the recovery period following each exercise event using the sensing system and the lactate concentrations measured therewith; and specifying, based on an output of the sensing system, that at least one of intensity, duration, or timing of a subsequent exercise event be modified to meet a specified training goal.

C. Methods for conducting or performing interval training. The methods comprise: directing or performing interval training in which a plurality of exercise events are conducted over a period of time; wherein at least a portion of the exercise events are conducted at an intensity such that a lactate threshold is exceeded; measuring a plurality of lactate concentrations in a biological fluid of an individual performing the interval training, the plurality of lactate concentrations being measured in vivo with a lactate-responsive sensor; wherein the plurality of lactate concentrations are measured at least while the lactate concentrations are decreasing in a recovery period following a peak lactate level reached in conjunction with each exercise event; communicating a signal from the lactate-responsive sensor to a processor located in a remote terminal or a local terminal; calculating, with the processor, a rate of lactate clearance during the recovery period following each exercise event, the rate of lactate clearance being based upon the plurality of lactate concentrations; and determining, with the processor, based upon the rate of lactate clearance, at least one of intensity, duration, or timing of a subsequent exercise event to meet a specified training goal.

C1. Methods for conducting or performing interval training. The methods comprise: measuring a plurality of lactate concentrations in a biological fluid of an individual performing interval training in which a plurality of exercise events are performed over a period of time and at least a portion of the exercise events are conducted at an intensity such that a lactate threshold is exceeded, the plurality of lactate concentrations being measured in vivo with a sensing system comprising a lactate-responsive sensor; wherein the plurality of lactate concentrations are measured at least while the lactate concentrations are decreasing in a recovery period following a peak lactate level reached in conjunction with each exercise event; communicating a signal from the lactate-responsive sensor to a processor located in a remote terminal, a local terminal, or a cloud server; calculating, with the processor, a rate of lactate clearance during the recovery period following each exercise event, the rate of lactate clearance being calculated using the plurality of lactate concentrations; and determining, with the processor and based upon the rate of lactate clearance, that at least one of intensity, duration, or timing of a subsequent exercise event be modified to meet a specified training goal.

D. Exercise methods. The methods comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining when a second exercise event should take place to meet a specified training goal based on the rate of lactate clearance.

D1. Exercise methods. The methods comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, when a second exercise event should take place to meet a specified training goal.

E. Exercise methods. The methods comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining an intensity at which a second exercise event should take place to meet a specified training goal based on the rate of lactate clearance.

E1. Exercise methods. The methods comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, an intensity at which a second exercise event should take place to meet a specified training goal.

F. Exercise methods. The methods comprise: directing or performing a first exercise event in which a lactate level above a baseline concentration has been reached, a recovery period following the first exercise event; measuring a plurality of lactate concentrations in a biological fluid of an individual performing the first exercise event, the lactate concentrations being measured over a period of time in the recovery period while the lactate concentrations are decreasing following a peak lactate level reached in conjunction with the first exercise event, the plurality of lactate concentrations being measured in vivo with a lactate-responsive sensor; calculating a rate of lactate clearance in the recovery period from the plurality of lactate concentrations; and determining how long a second exercise event should take place to meet a specified training goal based on the rate of lactate clearance.

F1. Exercise methods. The methods comprise: measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor; calculating a rate of lactate clearance in the recovery period using the sensing system and the plurality of lactate concentrations measured therewith; and specifying, based on an output of the sensing system and the rate of lactate clearance, how long a second exercise event should take place to meet a specified training goal.

G. Methods for determining physical fitness. The methods comprise: directing or performing an exercise event in which a lactate level above a baseline concentration has been reached, a recovery period following the first exercise event; measuring a plurality of lactate concentrations in a biological fluid of an individual performing the exercise event, the lactate concentrations being measured over a period of time in the recovery period while the lactate concentrations are changing, the plurality of lactate concentrations being measured in vivo with a lactate-responsive sensor; directing or performing a recovery activity following the exercise event, the recovery activity comprising no exercise or a prescribed level of exercise having an intensity less than that of the exercise event; determining a time from an end of the exercise event until a peak lactate level is reached; calculating a rate of lactate clearance in the recovery period from the plurality of lactate concentrations; and correlating the time until the peak lactate level is reached in the recovery period to a measure of physical fitness.

G1. Methods for determining physical fitness. The methods comprise: measuring a plurality of lactate concentrations in a biological fluid of an individual performing an exercise event in which a lactate level above a baseline concentration has been reached, the lactate concentrations being measured over a period of time in the recovery period while the lactate concentrations are changing, the plurality of lactate concentrations being measured in vivo with a sensing system comprising a lactate-responsive sensor; wherein a recovery activity follows the exercise event, the recovery activity comprising no exercise or a prescribed level of exercise having an intensity less than that of the exercise event; determining, based upon an output of the sensing system, a time from an end of the exercise event until a peak lactate level is reached; calculating a rate of lactate clearance in the recovery period using the sensing system and the plurality of lactate concentrations measured therewith; and correlating, based on the output of the sensing system, the time until the peak lactate level is reached in the recovery period to a measure of physical fitness.

Each of embodiments A-G and A1-G1 may have one or more of the following additional elements in any combination

Element 1: wherein a lactate threshold is exceeded in the first exercise event.

Element 2: wherein the peak lactate level is above the lactate threshold.

Element 3: wherein no exercise or a reduced level of exercise is conducted during the recovery period.

Element 4: wherein the predetermined concentration is at or above the baseline concentration.

Element 5: wherein the predetermined concentration is a set number, a percentage of the peak lactate level, or a multiple of the baseline concentration.

Element 6: wherein the method further comprises: determining a rate of lactate clearance in the recovery period from the plurality of lactate concentrations; and adjusting at least one of intensity, duration, or timing of the second exercise event in response to the rate of lactate clearance.

Element 6A: wherein the method further comprises: determining a rate of lactate clearance in the recovery period using the sensing system and the plurality of lactate concentrations; and specifying, based on the output of the sensing system and the rate of lactate clearance, at least one of intensity, duration, or timing of the second exercise event.

Element 7: wherein the rate of lactate clearance is determined by calculating a curve slope or a half-life of the lactate concentrations measured in the recovery period.

Element 8: wherein the method further comprises adjusting the rate of lactate clearance during the recovery period by conducting no exercise or a reduced level of exercise.

Element 8A: wherein the method further comprises: specifying, based on the output of the sensing system, that no exercise or a reduced level of exercise be performed during the recovery period to adjust the rate of lactate clearance.

Element 9: wherein the method further comprises: communicating a signal from the lactate-responsive sensor to a processor located in a remote terminal, a local terminal, or a cloud server; wherein the processor determines, based upon the plurality of lactate concentrations, a rate of lactate clearance and a training protocol selected from the group consisting of intensity of the second exercise event, duration of the second exercise event, timing of the second exercise event, and combinations thereof, and informs once the training protocol is available.

Element 10: wherein the method further comprises measuring a second plurality of lactate concentrations with the lactate-responsive sensor prior to reaching the peak lactate level.

Element 10A: wherein the plurality of lactate concentrations includes one or more lactate concentrations measured prior to reaching the peak lactate level.

Element 11: wherein a single lactate-responsive sensor measures the plurality of lactate concentrations over the period of time.

Element 12: wherein the lactate-responsive sensor comprises a working electrode that is insertable in a tissue.

Element 13: wherein the working electrode has a sensing region comprising a lactate-responsive enzyme disposed thereupon.

Element 14: wherein the lactate-responsive enzyme is covalently bonded to a polymer comprising the sensing region.

Element 15: wherein the lactate-responsive enzyme is lactate dehydrogenase or lactate oxidase.

Element 16: wherein the peak lactate level is above a lactate threshold and the subsequent exercise event is conducted after the lactate level has fallen below the lactate threshold.

Element 17: wherein the peak lactate level is below a lactate threshold and the subsequent exercise event is conducted after the lactate level has fallen to a predetermined concentration above the baseline concentration.

Element 18: wherein the subsequent exercise event is conducted after the lactate level has fallen to the baseline concentration.

Element 19: wherein the subsequent exercise event is conducted after the lactate level has fallen to a set number, a percentage of the peak lactate level, or a multiple of the baseline concentration.

Element 20: wherein the subsequent exercise event is conducted at a different intensity or duration than an initial exercise event in which the peak lactate level was reached.

Element 21: wherein a lactate threshold or the rate of lactate clearance changes between one or more of the exercise events.

Element 22: wherein the method further comprises adjusting the rate of lactate clearance during the recovery period by conducting no exercise or a reduced level of exercise.

Element 22A: wherein the method further comprises: specifying, based on the output of the sensing system, that no exercise or a reduced level of exercise be performed in the recovery period to adjust the rate of lactate clearance.

Element 23: wherein the method further comprises communicating a signal from the lactate-responsive sensor to a processor located in a remote terminal, a local terminal, or a cloud server; wherein the processor determines, based upon the plurality of lactate concentrations, the rate of lactate clearance and a training protocol selected from the group consisting of intensity of the subsequent exercise event, duration of the subsequent exercise event, timing of the subsequent exercise event, and combinations thereof, and informs once the training protocol is available.

Element 24: wherein the subsequent exercise event is conducted after the lactate concentrations have fallen below the lactate threshold.

Element 25: wherein the subsequent exercise event is conducted after the lactate concentrations have fallen to a set number, a percentage of the peak lactate level, a baseline concentration, or a multiple of the baseline concentration.

Element 26: wherein the subsequent exercise event is conducted at a different intensity or duration than an initial exercise event in which the lactate threshold was exceeded.

Element 27: wherein the lactate threshold or the rate of lactate clearance changes between one or more of the exercise events.

Element 28: wherein the method further comprises adjusting the rate of lactate clearance during the recovery period by conducting no exercise or a reduced level of exercise.

Element 28A: wherein the method further comprises: specifying with the processor that no exercise or a reduced level of exercise be conducted during the recovery period to adjust the rate of lactate clearance.

Element 29: wherein a lactate threshold is exceeded in the first exercise event.

Element 30: wherein the peak lactate level is above the lactate threshold.

Element 31: wherein the second exercise event is conducted after the lactate concentrations have fallen to a predetermined concentration that is at or above the baseline concentration.

Element 32: wherein the method further comprises adjusting intensity or duration of the second exercise event in response to the rate of lactate clearance.

Element 32A: wherein the method further comprises: specifying, based on the output of the sensing system, an adjustment of intensity or duration of the second exercise event in response to the rate of lactate clearance.

Element 32B: wherein the method further comprises: specifying, based on the output of the sensing system, an adjustment of timing or duration of the second exercise event in response to the rate of lactate clearance.

Element 32C: wherein the method further comprises: specifying, based on the output of the sensing system, an adjustment of intensity or timing of the second exercise event in response to the rate of lactate clearance.

Element 33: wherein the method further comprises communicating a signal from the lactate-responsive sensor to a processor located in a remote terminal, a local terminal, or a cloud server; wherein the processor determines, based upon the plurality of lactate concentrations, the rate of lactate clearance and a training protocol selected from the group consisting of intensity of the second exercise event, duration of the second exercise event, timing of the second exercise event, and combinations thereof, and informs once the training protocol is available.

Element 34: wherein no exercise or a reduced level of exercise is conducted during the recovery period.

Element 35: wherein the rate of lactate clearance is determined by calculating a curve slope or a half-life of the lactate concentrations measured in the recovery period.

Element 36: wherein the method further comprises: adjusting the rate of lactate clearance during the recovery period by conducting no exercise or a reduced level of exercise.

Element 36A: wherein the method further comprises: specifying, based on the output of the sensing system, that no exercise or a reduced level of exercise be conducted during the recovery period.

Element 37: wherein the rate of lactate clearance is determined by calculating a curve slope or a half-life of the lactate concentrations measured in the recovery period, or a predetermined decrease in the lactate concentrations.

Element 38: wherein the curve slope or the half-life of the lactate concentrations, or the predetermined decrease in the lactate concentrations defines the measure of physical fitness.

By way of non-limiting example, exemplary combinations applicable to A and A1 include: 1 and 3; 3 and 4; 4 and 5; 3 and 5; 4 and 6 or 6A; 1 and 6 or 6A; 6 or 6A and 7; 6 or 6A and 8 or 8A; 6 or 6A and 9; 1 and 11; 3 and 11; 4 and 11; 6 or 6A and 11; 8 or 8A and 11; 11 and 12; and 13 and 14. Exemplary combinations applicable to B and B1 include: 16 and 18; 17 and 18; 16 and 19; 17 and 19; 18 and 20; 19 and 20; 18 and 21; 19 and 21; 18 and 22 or 22A; 19 and 22 or 22A; 21 and 23; 22 or 22A and 23; 16 and 23; and 17 and 23. Exemplary combinations applicable to C and C1 include: 24 and 26; 24 and 27; 25 and 27; 25 and 26; 26 and 27; 24 and 28 or 28A; 25 and 28 or 28A; 26 and 28 or 28A; and 27 and 28 or 28A. Exemplary combinations applicable to D-G and D1-G1 include: 28 and 31; 29 and 31; 31 and 32, 32A, 32B or 32C; 31 and 33; 32, 32A, 32B or 32C and 33; 31 and 34; 32, 32A, 32B or 32C and 34; 31 and 35; 34 and 35; 33 and 35; 31 and 36 or 36A; 32, 32A, 32B or 32C and 36 or 36A; 33 and 36 or 36A; 34 and 36 or 36A; 35 and 37; 35 and 38; and 37 and 38.

To facilitate a better understanding of the embodiments described herein, the following examples of various representative embodiments are given. In no way should the following examples be read to limit, or to define, the scope of the invention.

### EXAMPLES

A FREESTYLE LIBRE™ (Abbott Diabetes Care) sensor housing was fitted with a lactate-responsive sensor, and lactate levels were monitored continuously over the course of several hours to several days. The relative time was recorded when each lactate concentration measurement was made. Various analyses were conducted as described hereinafter.

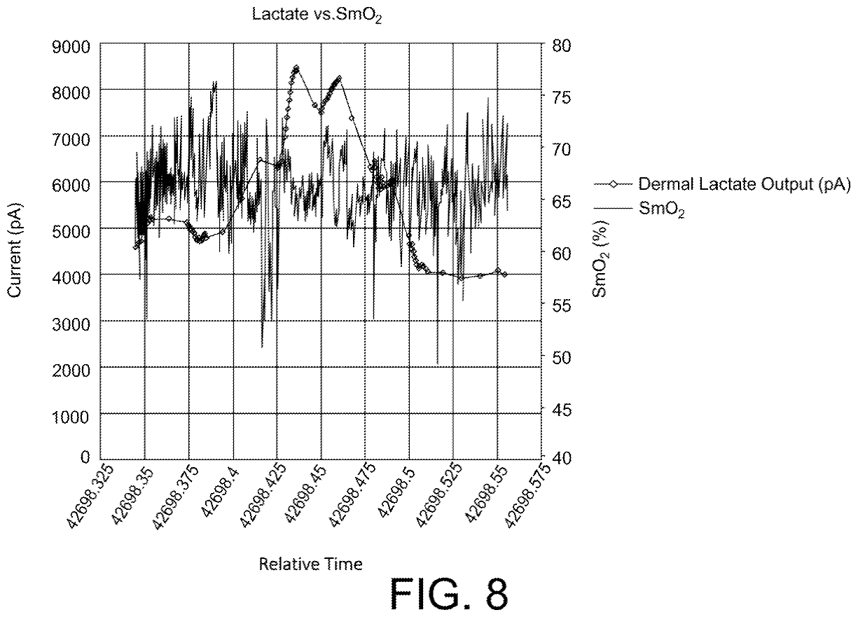

FIG. 8 shows the output of a lactate-responsive sensor as compared to lactate concentrations measured via an external infrared spectroscopy technique (BSX or Moxy monitor). As shown, the infrared-determined lactate concentrations were much more variable than were the sensor-determined lactate concentrations. Moreover, the lactate concentrations determined by the two methods did not track especially well with one another.

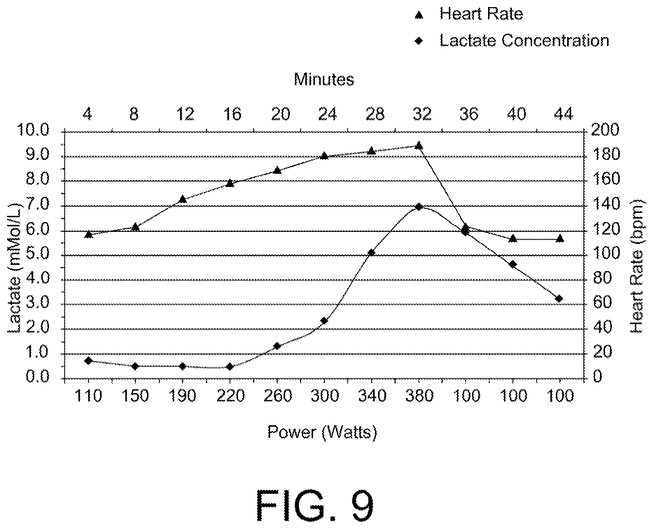

FIG. 9 shows the output of a lactate-responsive sensor as compared to heart rate. As shown, heart rate tended to exhibit a more linear response to increased intensity, while the lactate concentration tended to change slope at particular inflection points

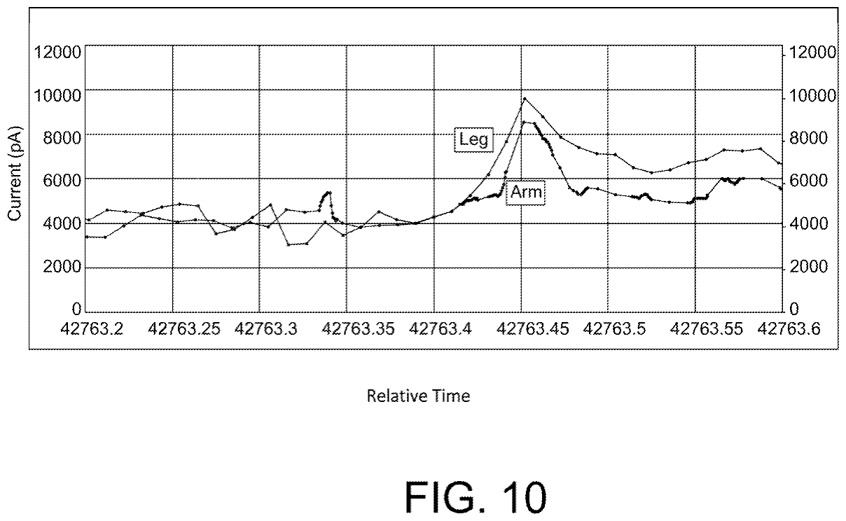

FIG. 10 shows the output of two different lactate-responsive sensors worn in different locations during a cycling event. One of the sensors was worn on the upper arm, and the other was worn on the leg. As shown, the lactate concentrations measured with the sensor worn upon the leg were slightly higher. The higher lactate concentrations measured with the sensor worn upon the leg are indicative of the greater work performed by the leg muscles during cycling. The lactate concentrations measured at each location approximately tracked each other.

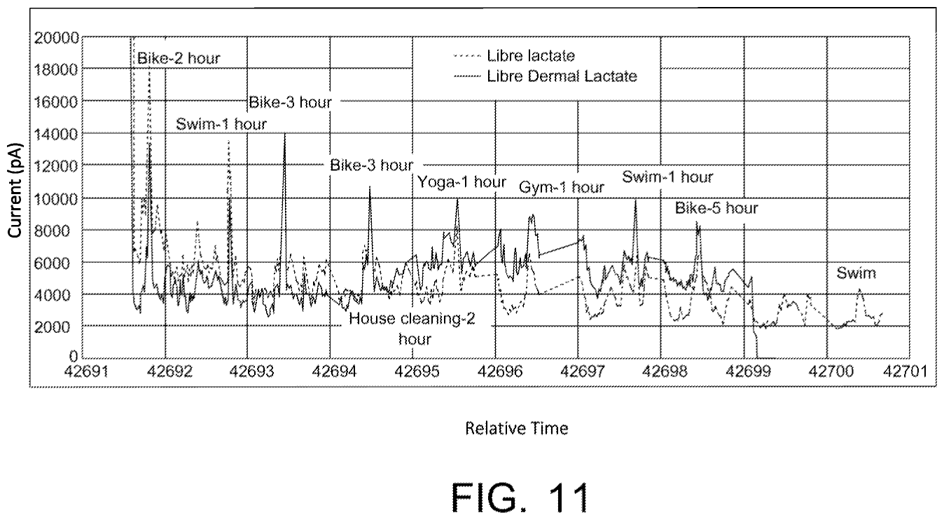

FIG. 11 shows the lactate sensor response as a function of time for two different lactate-responsive sensors implanted at different depths. One sensor was worn on the left arm, and the other was worn on the right. Again, the measured lactate concentrations were not identical, but they approximately tracked each other.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

1. A method comprising:
measuring a plurality of lactate concentrations in a biological fluid in vivo with a sensing system comprising a lactate-responsive sensor over at least a period of time while the lactate concentrations are decreasing following a peak lactate level reached in conjunction with a first exercise event, a lactate level above a baseline concentration being reached in the first exercise event; and
specifying, based on an output of the sensing system, that a second exercise event be conducted after a recovery period in which the lactate level has fallen to a predetermined concentration, the recovery period interceding between the first exercise event and the second exercise event.

2. The method in claim 1, wherein a lactate threshold is exceeded in the first exercise event.

3. The method of claim 2, wherein the peak lactate level is above the lactate threshold.

4. The method in claim 1, wherein no exercise or a reduced level of exercise is conducted during the recovery period.

5. The method of claim 1, wherein the predetermined concentration is at or above the baseline concentration.

6. The method of claim 5, wherein the predetermined concentration is a set number, a percentage of the peak lactate level, or a multiple of the baseline concentration.

7. The method of claim 1, further comprising:
determining a rate of lactate clearance in the recovery period using the sensing system and the plurality of lactate concentrations; and
specifying, based on the output of the sensing system and the rate of lactate clearance, at least one of intensity, duration, or timing of the second exercise event.

8. The method in claim 7, wherein the rate of lactate clearance is determined by calculating a curve slope or a half-life of the lactate concentrations measured in the recovery period.

9. The method in claim 7, further comprising:
specifying, based on the output of the sensing system, that no exercise or a reduced level of exercise be performed during the recovery period to adjust the rate of lactate clearance.

10. The method of claim 1, further comprising:
communicating a signal from the lactate-responsive sensor to a processor located in a remote terminal, a local terminal, or a cloud server;
wherein the processor determines, based upon the plurality of lactate concentrations, a rate of lactate clearance and a training protocol selected from the group consisting of intensity of the second exercise event, duration of the second exercise event, timing of the second exercise event, and combinations thereof, and informs once the training protocol is available.

11. The method of claim 1, wherein the plurality of lactate concentrations includes one or more lactate concentrations measured prior to reaching the peak lactate level.

12. The method of claim 1, wherein a single lactate-responsive sensor measures the plurality of lactate concentrations over the period of time.

13. The method of claim 1, wherein the lactate-responsive sensor comprises a working electrode that is insertable in a tissue.

14. The method of claim 13, wherein the working electrode has a sensing region comprising a lactate-responsive enzyme disposed thereupon.

15. The method of claim 14, wherein the lactate-responsive enzyme is covalently bonded to a polymer comprising the sensing region.

16. The method of claim 14, wherein the lactate-responsive enzyme is lactate dehydrogenase or lactate oxidase.

17.-31. (canceled)

32. A method comprising:
measuring a plurality of lactate concentrations in a biological fluid of an individual performing a first exercise event in which a lactate level above a baseline concentration has been reached and a recovery period follows the first exercise event, the lactate concentrations being measured over a period of time in the recovery period while the lactate concentrations are decreasing following a peak lactate level reached in conjunction with the first exercise event, the plurality of lactate concentrations being measured in vivo with a sensing system comprising a lactate-responsive sensor;
calculating a rate of lactate clearance in the recovery period using the sensing system and the plurality of lactate concentrations measured therewith; and
specifying, based on an output of the sensing system and the rate of lactate clearance, at least one of a start time of a second exercise event to meet a specified training goal, an intensity at which the second exercise event should take place to meet the specified training goal, or a duration of the second exercise event should take place to meet the specified training goal.

33. The method of claim 32, wherein a lactate threshold is exceeded in the first exercise event.

34. The method of claim 33, wherein the peak lactate level is above the lactate threshold.

35. The method of claim 32, wherein the second exercise event is conducted after the lactate concentrations have fallen to a predetermined concentration that is at or above the baseline concentration.

36. (canceled)

37. The method of claim 32, further comprising:
communicating a signal from the lactate-responsive sensor to a processor located in a remote terminal, a local terminal, or a cloud server;
wherein the processor determines, based upon the plurality of lactate concentrations, the rate of lactate clearance and a training protocol selected from the group consisting of the intensity of the second exercise event, the duration of the second exercise event, the start time of the second exercise event, and combinations thereof, and informs once the training protocol is available.

38. The method of claim 32, wherein no exercise or a reduced level of exercise is conducted during the recovery period.

39. The method of claim 32, wherein the rate of lactate clearance is determined by calculating a curve slope or a half-life of the lactate concentrations measured in the recovery period.

40. The method of claim 32, further comprising:
specifying, based on the output of the sensing system, that no exercise or a reduced level of exercise be conducted during the recovery period.

41.-61. (canceled)
